(19) United States
(12) Patent Application Publication
(10) Pub. No.: US 2008/0269792 A1
(43) Pub. Date: Oct. 30, 2008

(54) Needle Device and Methods of Use

(76) Inventor: Herbert Warren Collins, (US)

Correspondence Address: Pamela M. Guy, 31 Daniels Way, Boxborough, MA, 01719, US

(21) Appl. No.: 11/739,352

(22) Filed: Apr. 24, 2007

(51) Int. Cl. A61B 17/34

(52) U.S. Cl. 606/189

(57) ABSTRACT

The technology described herein relates to needle devices and related methods. In one aspect, the technology provides a needle device that includes a rotator portion comprising a plurality of needles and a handle portion. The needles can be arranged according to a particular use, and are generally arranged in one or more rows. In various embodiments, the needles are spaced about 6 mm apart from each other, less than about 6 mm apart from each other and more than about 6 mm apart from each other. In various embodiments, the needles can be present in one row or more than one row. In an embodiment, the needles are present on a disposable strip. Also described are a needle device comprising a plurality of needles present on a planar support, in which the needles are arranged into one or more rows, and related methods.

### BACKGROUND

The nervous system of the body plays an important role in the outcome of non-fatal injury, even when no structures of the nervous system are directly injured. Historical records reaching back as long ago as three thousand BC suggest that placement of needles into the skin of an individual has been used for therapeutic benefit. For instance, the body of the Italian iceman recovered in 1990 had tattoos suggesting therapeutic treatment of lower extremity discomfort. From what is now known, it appears that the Iceman's Shaman likely used aspects of neurophysiology to determine locations where tattoos would provide symptomatic relief. The Iceman's skin had three lines of tattoos that were unilateral, parallel, and adjacent to the lumbar spine. One of these lines probably recorded the first time the Iceman obtained relief of his lower extremity discomfort. The other two lines may represent the Iceman's return for relief of recurrent symptoms.

In the 1940s a Russian physician is credited with being the first to provide evidence that, in the presence of survivable anatomical insults, death is an expression of neurophysiology. Specifically, a person having a survivable injury can be rendered without pulse or breath due to the neurophysiology expressed in his clinical picture. The interruption of this neurophysiology due to a brief period of abnormally reduced oxygen content in the blood (anoxemia) allows some patients to be revived. Later on it was demonstrated that electric shock therapy had the same affect as anoxemia, in terms of allowing patient revival. It was shown that resuscitative efforts of this type had saved many lives, although survivors unfortunately had varying degrees of brain damage. Autopsy cannot identify patients who had survivable insults yet died of brain damage and therefore the number and percentage of failed resuscitative efforts is unknown.

The role of the nervous system in the outcome of non-fatal injury was also shown in the 1970s by published work on treatment of Herpes zoster by sublesional corticosteroid injections. It was reported that patients responded to these injections with an augmented burst of pain, while injections at adjacent sites produced a more muted pain response. After the completion of these injections some patients reported pain relief, and certain of them reported relief of a deep-seated discomfort that they were not aware of until it was relieved.

A more widely recognized medical intervention involving needle pricks is acupuncture, which apparently was studied as early as 2000 BC in China. According to legend, a Chinese physician became intrigued by soldiers' accounts of disappearance of symptoms that had plagued them for years following minor battlefield wounds. Meticulous notes were followed and weapons were used to create wounds at specific sites to relieve patients suffering similar symptoms. Later it was learned that skin pricks were as effective as deeper wounds.

Another medical intervention involving needle pricks is non-chemical, non-electrical intramuscular stimulation (IMS), which has been used for treating myofascial pain (fibromyalgia). During IMS generally one pin is used at a time. The pin, which is inserted into a tender muscle motor point, is continuously manipulated to achieve pain relief (see, for example, Gunn et al Spine, 5(3): 279-291 (1980)). Gunn et al. have described that IMS treatment is quite painful to the patient, and that this pain is primarily due to the irregular deflection of the needle from its proper path as it is manually pushed in and pulled out repetitively through tissues of differing resiliencies.

Other recent studies have shown that neurological processing can by favorably modulated by administering appropriate distribution specific needle skin pricks. For example, Collins, H. (Med Hypo 50:417-419(1998)) describes use of therapeutic distribution specific needle pricks to change the value assigned to a nervous system impulse, when such an impulse is altered by the presence of a distribution specific anatomical insult. In theory, the value of a nervous system impulse of a specific spinal nerve distribution is inversely related to the magnitude of normal cutaneous impulses corresponding to contact of the skin with the external environment. Application of spinal nerve specific cutaneous skin pricks results in a change in the value of a nervous system impulse, resulting in the value becoming too low to meet the threshold necessary for continued expression in the clinical picture. Theory aside, this publication demonstrates that cutaneous needle pricks can be used to beneficially alter neurophysiology and provide relief to individuals experiencing a variety of clinical pictures.

### SUMMARY

The technology described herein relates to needle devices and related methods. In one aspect, the technology provides a needle device that includes a rotator portion comprising a plurality of needles and a handle portion. The needles can be arranged according to a particular use, and are generally arranged in one or more rows. In various embodiments, the needles are spaced about 6 mm apart from each other, less than about 6 mm apart from each other and more than about 6 mm apart from each other. In various embodiments, the needles can be present in one row or more than one row. In an embodiment, the needles are present on a disposable strip. In another aspect, the technology provides a needle device including a plurality of needles present on a planar support, wherein the needles are arranged into one or more rows. In various embodiments, the needles are spaced about 6 mm apart from each other, less than about 6 mm apart from each other and more than about 6 mm apart from each other. In various embodiments, the needles can be present in one row or more than one row. In an embodiment, the needles are present on a disposable strip.

Provided by the technology described herein are methods for using the described needle devices. In one aspect, a method is provided for treating an individual having an anatomical insult. The method involves delivering multiple cutaneous needles pricks to skin overlying an area innervated by sensory fibers of one or more nerve roots using a needle device described herein. In another aspect, a method is provided for treating pain in an individual. The method involves delivering multiple cutaneous needle pricks to skin overlying an area innervated by sensory fibers of one or more nerve roots using a needle device described herein, whereby pain is reduced. In a further aspect, a method is provided for diagnosing an individual experiencing pain. The method involves delivering multiple cutaneous needle pricks to skin overlying an area innervated by sensory fibers of one or more nerve roots using a needle device described herein, and correlating a pain response at a specific site of skin overlying an area innervated by sensory fibers of one or more nerve roots with an area of the body.

### BRIEF DESCRIPTION OF DRAWINGS

FIGS. 1 A, B and C show an exemplary needle device having a plurality of needles displayed on a rotator.

FIG. 2 shows an exemplary needle device having a plurality of needles displayed on a planar support.

### DETAILED DESCRIPTION

The technology described herein relates to needle devices and related methods. The needle devices can be used, for example, for beneficially modulating the neurophysiology of specific spinal nerve distributions. Although the specific mechanism of the therapeutic methods remain uncertain, the inventor has observed that cutaneous needle pricks can alter neurological processing and provide a variety of therapeutic benefits. Clinical results, some of which are presented in the Case Reports below, have shown the methods to be highly effective and to exhibit no appreciable undesirable side effects.

The needle devices described herein allow a practitioner to deliver multiple needle pricks without the need for individual placement of needles. By avoiding individual placement of needles, time of therapy can be reduced, pain experienced by the individual undergoing treatment can be reduced, and risk of practitioner accidental needle sticks is reduced. In addition, the depth and placement of the needle pricks can be controlled using a needle device described herein.

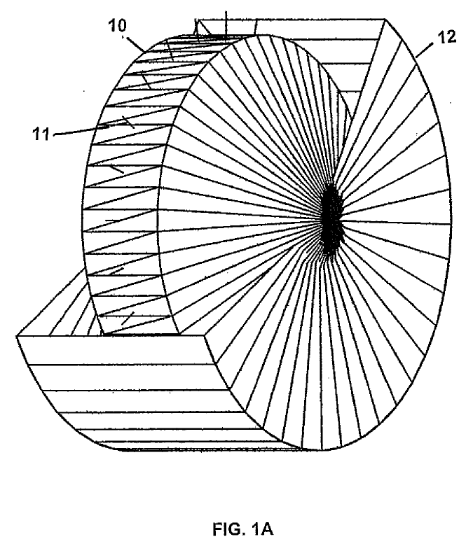

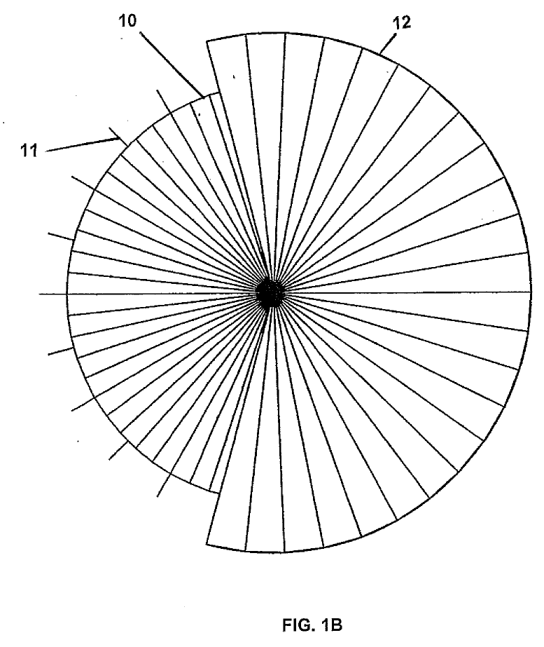

FIG. 1 depicts an exemplary needle device including a rotator portion 10 comprising a plurality of needles (single needle shown as 11), and a handle portion 12. The rotator portion 12 functions to position multiple needles in an arrangement whereby rolling the rotator upon the skin of an individual results in needle penetration into the skin at a selected penetration depth. The needles are spaced on the rotator to provide a desired pattern of needle pricks to the skin, depending on the therapeutic or diagnostic procedure and size of the individual to receive treatment. The rotator portion 12 typically has a diameter of about 25-50 mm or any convenient size that allows multiple needles to be presented. In an embodiment, one row of needles is presented. In another embodiment, two rows of needles are presented. In an embodiment, at least two rows of needles are presented. The number of needles presented on the rotator can vary. The distance between needles also can vary depending on the desired pattern of cutaneous needle pricks. In a specific embodiment, the needles are spaced about 6 mm or further apart from each other. In an embodiment, the needles are spaced less than about 6 mm apart from each other. Needles can be spaced evenly or unevenly in any suitable pattern expected to produce a desired therapeutic or diagnostic result.

The handle portion 12 although shown in FIG. 1. as an arc-shape, can have any design that allows for holding the device and guiding it along the skin of an individual. The handle portion 12 can thus have different sizes and shapes suitable for different practitioners; can be textured, for example to enhance gripping by a practitioner; can be constructed to provide desired weight of the device; can be weighted to achieve a particular balance; and can be constructed from various materials, such that the device can be sterilized, resistant to chemical reagents, disposable, and the like.

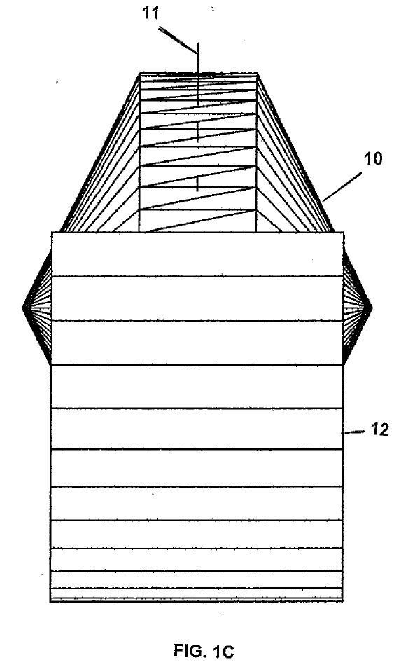

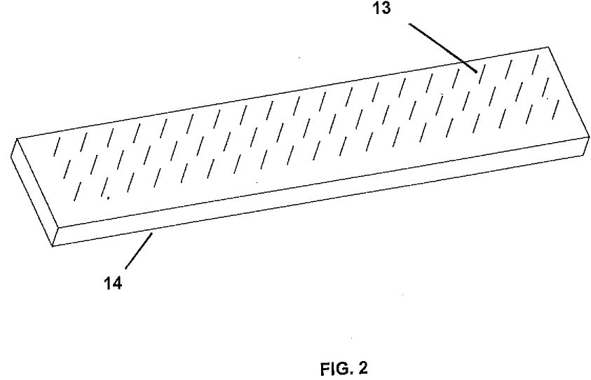

In another aspect, the technology provides a needle device including a plurality of needles present on a planar support. FIG. 2 depicts an exemplary needle device including a plurality of needles (single needle shown as 13) present on a planar support 14. The needles can be spaced in any manner expected to produce a desired therapeutic or diagnostic result. In an embodiment, the needles are spaced about 6 mm apart from each other. In an embodiment, the needles are spaced less than about 6 mm apart from each other. In various embodiments, the plurality of needles can be present in one row, two rows and more than two rows. Needles can be spaced evenly or unevenly in any suitable pattern expected to produce a desired therapeutic or diagnostic result. In a further embodiment, the plurality of needles is present on a disposable strip. The planar support 14 can have any design that allows for holding the device and guiding it along the skin of an individual. The planar support can be block-shaped as is shown in FIG. 2, or have a curved shape. For example, the planar support can be shaped for convenient or comfortable gripping by a practitioner. As such, the planar support can have different sizes and shapes suitable for different practitioners; can be textured, for example to enhance gripping by a practitioner; can be constructed to provide desired weight of the device; can be weighted to achieve a particular balance; and can be constructed from various materials, such that the device can be sterilized, resistant to chemical reagents, disposable, and the like.

Needles useful for a needle devise described herein can be hollow or solid, having a size corresponding generally to a mid to large gauge, such as between about 23 gauge and about 27 gauge, for example 25 gauge, referring to Stubs needle gauging nomenclature. A larger gauge needle can be selected to increase the level of stimulation associated with treatment. To avoid stimulating structures deeper than the skin, the needles typically are inserted to a depth of about 1 mm. Thus, the length of needles is typically a fraction of a cm, for example about 1 mm, 5 mm, 10 mm, 20 mm or larger. The needles can present a substance, such as a medicament, if desired.

The needle devices described herein include a plurality of needles present on a support, such as rotator and planar supports. The needles can be provided in a strip format that can be attached to the support and later removed for disposal. Attachment can be achieved by mechanical and chemical means, such as clips, snaps, adhesive, hook and loop-type fasteners and the like.

It is standard medical practice that needles used for treating individuals are sterile. Practitioners should use a new strip of disposable needles taken from a sealed package for each individual and should swab treatment sites with alcohol or another disinfectant before inserting needles. Accordingly, a needle device of the invention, or a portion thereof, such as a needle strip, needle presenting support, such as the rotator or planar support described herein, and/or handle portion can be sterile and provided in sterile packaging. Alternatively or additionally, the needle device or portion thereof can be constructed from autoclavable or otherwise sterilizable material. It will be recognized by medical practitioners that the needle devices described herein can be used for a variety of purposes in addition to the exemplary therapeutic and diagnostic methods described herein, such as allergy testing in which various potential allergens are presented on the needle devices.

Provided by the technology described herein are methods for using the described needle devices. In one aspect, a method is provided for treating an individual having an anatomical insult. The method involves delivering multiple cutaneous needles pricks to skin overlying an area innervated by sensory fibers of one or more nerve roots using a needle device described herein. In another aspect, a method is provided for diagnosing an individual experiencing pain. The method involves delivering multiple cutaneous needle pricks to skin overlying an area innervated by sensory fibers of one or more nerve roots using a needle device described herein and correlating a pain response at a specific site of skin overlying an area innervated by sensory fibers of one or more nerve roots, with an area of the body. A location of injury can be determined based on an increased level of patient's pain response when needle pricks are delivered to the skin overlying an area innervated by sensory fibers of one or more nerve roots. An increased pain response generally correlates with the location of sensory fibers related to the bodily injury. Using any of a variety of known maps which show linkages of specific areas of the body with specific sensory fiber bundles, the injured area of the body, as determined by needle pricks, can be identified.

In the methods described herein, needles presented on needle devices are used to prick skin overlying an area innervated by sensory fibers of one or more nerve roots. For the sake of background information, the spinal cord can be divided into segments according to the nerve roots that branch off of it. Nerves along the cord consists of 7 cervical, 12 thoracic, 5 lumbar, 5 sacral, and 1 coccygeal nerve. The nerve roots run through the bony canal, and at each level a pair of nerve roots exits from the spine. In the cervical spine (neck), the nerve root in common convention is named for the lower segment that it runs between (for example, the nerve root at C5-C6 segment is termed the “C6 nerve root”). In the lumbar spine (lower back), the nerve is named for the upper segment that it runs between (for example, the nerve root at the L4-L5 segment is termed the “L4 nerve root”). The term “dermatome” has been used to describe a segment of a human body innervated by a single dorsal root. Dermatomes follow a highly regular pattern on the body and are categorized into four major regions, the cervical (C), the thoracic (T), the lumber (L) and the sacral (S) regions. Diagrams of distributions of skin innervations are widely available, and can be used by a practitioner to select regions for treatment based on the symptoms or condition of the individual to be treated.

To identify a spinal nerve responsible for monitoring insults within its specific anatomical distribution, which is appropriate for the particular condition of an afflicted individual, an initial needle prick is delivered to the area of the suspected spinal nerve. If the placement of the initial needle prick produces an exaggerated pain response, needle pricks are then extended in a linear fashion both in caudal and cephalic directions until a more muted response is encountered. When treating visceral insults, appreciable symptomatic relief is typically achieved once needle pricks are delivered over the roots of all respective bilateral pairs of spinal nerves. However, some visceral distributions are as follows: pharyx at bilateral C-3; cardiac at bilateral T3-4; gastric at bilateral T6-7; duodenal and renal at bilateral L3-4.

Assume for the sake of explanation that a source of pain is located in the lumbar region L3-4. The individual would be arranged face down in a comfortably relaxed manner. Needle pricks would be applied to his back, beginning with the L3-4 region, to locate an area that produces an exaggerated pain response. Needle pricks would then be extended outwards until a muted pain response is exhibited.

If the individual was experiencing pain at an unknown location, such as visceral pain, which is typically of unknown origin, needle pricks also would be applied to his back at locations of roots of all spinal nerves. Like the previous example in which the source of pain is known, once an area that produces an exaggerated pain response is identified, needle pricks would be extended outwards until a muted pain response is exhibited. Taking the example of an individual experiencing visceral pain and having exaggerated pain response at bilateral T6-7, such a pain response would be correlated with a gastric condition or ailment.

Studies described in Collins, H. (Med Hypo 50:417-419 (1998)) show that specific documented visceral insults are correlated with the patient's response to bilateral distribution specific skin pricks. The ability of skin pricks to correlate cutaneous stimulation uniquely to specific distributions allows a practitioner to map unknown but specific visceral distributions. Such correlating of visceral pathology to known specific visceral distributions will facilitate selection of diagnostic studies to identify or confirm a specific anatomical insult.

Methods for providing medical treatment using cutaneous needles pricks to skin overlying an area innervated by sensory fibers of one or more nerve roots has been described previously. For example, Collins, H. Med. Hypo 50:417-419 (1998) describes case reports in which needle pricks were used to provide benefit to patients inflicted with a variety of medical concerns. These case reports, summarized below, are examples of appropriate scenarios for using a needle prick device of the invention described herein. The intradermal injections described in these reports can be replaced with needle pricks without loss of efficacy. Whereas the previous methods were performed using conventional individually applied needles, the needle prick devices described herein can allow for more rapid delivery of needle pricks at desired intervals.

The above described needle prick methods can be combined with exercises and various therapeutic modalities as appropriate for the condition being treated.

Although not wishing to be bound by theory, the paragraphs below, preceding the Case Studies, express the inventor's hypothesis concerning the efficacy of needle prick therapy.

With the exception of the central nervous system independent segments of both anatomy and neurophysiology are defined by the specific cutaneous, somatic, and visceral distributions of spinal nerves. The viscera of the mid-gut receives specific and independent visceral distributions contributed by bilateral pairs of spinal nerves. Unless anatomical insults represent a structural or physiological compromise they contribute little to the clinical picture. For the most part clinical pictures are neurological expressions. Neurophysiology expressed in clinical pictures originates from the results of the processing that occurs uniquely and within the specific distributions of individual spinal nerves. Impulses altered by the presence of distribution specific anatomical insults are assigned processed values that are inversely related to impulses generated through the skin's contact with the external environment. When altered impulses are processed within the equation containing a relatively low level of cutaneous impulses then assigned processed values become relatively significant. Relatively significant values exceed the threshold necessary and are expressed in the clinical picture. However when processed within the equation containing an exaggerated level of cutaneous impulses assigned processed values become relatively insignificant. Relatively insignificant values are too low to gain further expression in the clinical picture. This interruption of neurological expressions like that accomplished by anoxemia, and/or electric shock is more or less permanent. The long term duration of this symptomatic relief escapes explanation. The neurophysiology expressed in clinical pictures is much more than that of patient discomfort. These expressions are also that of physiological and even anatomical changes. These changes produce their own neurological expressions. These secondary neurological expressions account for the perpetuation nature of the neurophysiology expressed in clinical pictures. In the presence of initially survivable anatomical insults as that of surgery, infections, trauma, infarctions, or others it might take this perpetuating neurophysiology moments, hours, or days to render a patient to be without pulse or breath. Now one must wager that before the onset of terminal brain damage the depressing effects of anoxemia, and/or electric shock therapy will interrupt the neurological expression of death. All who survive this sometimes unwarranted ordeal will have varying degrees of brain damage. Some damage will be so severe that independent living is impossible.

Autopsy findings can not identify Individual also with survivable insults yet who suffers terminal brain damage. Therefore the number or percentage of failed resuscitative efforts is unknown. Preserving survival potential and preventing unwarranted brain damage hinges on the ability to interrupt the neurophysiology expressed in clinical pictures at the onset of therapy. Visceral insults generate neurophysiology that lacks information that pin points the exact anatomical location of the insult. Consequently the presence of specific visceral distributions has been discounted in the past. However the viscera a product of the mid-gut receives specific and independent distributions contributed by bilateral pairs of spinal nerves. For this reason no appreciable symptomatic relief will occur unless the relativity of the neurological processing of both of the paired spinal nerves has been exploited. When specific and documented visceral insults are correlated with the relative level of the neurological activity of specific pairs of spinal nerves no variations are found.

During the research phase care was taken to be sure an abundance of uniquely specific distribution cutaneous impulses were generated and at the same time efforts were made not to involve deeper structures. For this reason intradermal blebs of saline were used to generate cutaneous impulses that would be confined only to the respective and specific distributions. However bloodless needle pricks are just as effective. Exploratory needle pricks are placed unilaterally, adjacent, and parallel to the spinous process. Once the patient demonstrates an augmented painful response then needle pricks are extended in a cephalic and caudal direction until a more muted patient response is encountered. The identification of a spinal nerve monitoring anatomical insults is dependent on the patient experiencing an unmistaken augmented burst of pain in response to distribution specific needle pricks. This burst of pain accompanied by profound and long term symptomatic relief is interpreted as heralding the interruption of the neurophysiology formerly expressed in the clinical picture.

Bilateral pairs of spinal nerves contribute independent and separate specific visceral distributions. Therefore patients are not expected to experience appreciable symptomatic relief from visceral insults until the neurophysiology of both of the involved pair of spinal nerves has been addressed. For unknown reasons when abdominal visceral insults occupy more than one segment of neurophysiology no symptomatic relief will be appreciated until the neurophysiology of all of the respective bilateral pairs of spinal nerves at all of the sites have been addressed. This represents a fail safe mechanism which prevents even distant multiple sites of abdominal visceral insults from being overlooked. Interrupting the neurophysiology expressed in clinical pictures does not compromise the continued function of the nervous system. That is if the expression of the neurophysiology created in response to a myocardial infarction has been interrupted then extension of the infarct will cause an immediate resurgence of symptoms.

Physicians can not prevent death they can only reschedule death for an unknown future event and time. Patient insults can be arranged into three different degrees of risk. One is an insult that can not be survived under any circumstance. Another type of insults are those that will be survived even without intervention. Then there is a third degree of insults that can be survived only if anatomical insults are isolated from the neurophysiology currently expressed in the clinical picture. At the present time there are two roads to this isolation. One is to allow this neurophysiology to perpetuate until the patient has become to be without pulse or breath. Then hopefully the patient will survive a resuscitative effort. The other road is to interrupt the neurophysiology currently expressed in clinical pictures at the onset of therapy and at the spinal nerve level.

Little has changed since WWII when a Russian Physician provided proof that the neurophysiology expressed in clinical pictures is capable of rendering an individual with survivable anatomical insults to be without pulse or breath. Survivable insults are identified if resuscitative efforts are successful. It is generally accepted that if resuscitative efforts are not successful then survivable potential was not present. However autopsy findings can not identify those also with survivable insults yet who suffer terminal brain damage. Therefore the number or percentage of failed resuscitative efforts is unknown. Benefits of resuscitative efforts encouraged the belief that this effort saved more lives than any other procedure. At the same time this exuberance led physicians to remain oblivious and complacent of the carnage associated with preventable, delayed, or failed resuscitative efforts.

The ability to select the time and the route of interrupting the neurophysiology expressed in clinical pictures removes this effort from the theater of urgency. This interruption is dependent on exploiting the relativity of the equation at the spinal nerve level where impulses altered by the presence of anatomical insults are assigned processed relative values. Relative processed values are related inversely to the quantity of cutaneous impulses usually generated through the skins contact with the external environment. These processed relative values can be reduced to insignificant values by exploiting the relativity of the processing equation.

This can be accomplished by the purposeful generation of an excess of cutaneous impulses. The generation of an excess of distribution specific cutaneous impulses can best be accomplished by distribution specific bloodless needle pricks. An excess of cutaneous impulses result in the reduction of the relativity of assigned processed values to ones that are too low to gain further expression in the clinical picture. The ability to select the time and place for interrupting the neurophysiology expressed in clinical pictures allows one to circumvent much of the carnage associated with resuscitative efforts. Timely interruption at the onset of therapy not only promises a convalescence that is safer but also ones that are more comfortable and are associated with a lesser degree of disability.

Bloodless needle pricks are a time honored method of generating cutaneous impulses. They not only generate an abundance of cutaneous impulses but also this generation is uniquely confined to selected distributions. The ability to confine the generation of cutaneous impulses to specifically selected distributions is important for diagnostic purposes but also for the mapping of the unknown but specific visceral distributions.

### Case Report 1

This case report provides an example of needle prick treatment for pain relief. The patient was a healthy young automotive service station attendant. His hand had been burned when he removed the cap from a hot radiator. To reduce the patient's pain, intradermal blebs of saline were placed in the area overlaying the roots of the respective unilateral spinal nerves. After an exaggerated pain response, the patient experienced profound symptomatic relief. This rapid onset of pain relief produced a brief shock-like state that was alleviated by elevation of the feet. After appropriate dressings were applied to the injury, the patient returned to work without complication. With the resultant reduction in discomfort and lack of substantial bullae and edema formation observed after this cutaneous needle prick treatment, his recovery was more rapid than expected.

### Case Report 2

This case report provides an example of needle prick therapy for treating Herpetic lesions. Before the advent of antiviral therapy an elderly man presented with a prominent case of Herpetic lesions involving the ophthalmic branch of the trigeminal nerve. A one time saline infiltration at unilateral C2 resulted in complete symptomatic relief, with healing completed in five days, and without scarring or post Herpetic pain.

### Case Report 3

This case report provides an example of needle prick treatment for treating sore throat. A college student had a sore throat so severe that he was unable to swallowing his saliva. Intradermal saline infiltrations were made over the roots of bilateral C3, producing exaggerated discomfort immediately followed by profound symptomatic relief. He then drank over a liter of water and experienced a rapid recovery without further discomfort or difficulty in swallowing. Laboratory findings received later were compatible with the diagnosis of infectious mononucleosis.

### Case Report 4

This case report provides an example of needle prick therapy for cardiovascular associated pain relief. In spite of maximum doses of IV morphine over a period of twenty four hours, a patient with a myocardial infarction experienced no appreciable symptomatic relief. Bilateral intradermal saline infiltration at T3-4 produced an exaggerated pain response followed by profound symptomatic relief. He had an uneventful recovery without further pain or discomfort.

### Case Report 5

This case report provides an example of needle prick therapy for post-surgical pain after a ruptured duodenal ulcer. The patient was a housewife experiencing persistent severe discomfort to the extent that she was unable to eat or adequately sleep. Intradermal saline infiltration at bilateral L3-4 produced immediate relief and improvement of her previously haggard appearance. Within the next 24 hours, she had been eating and sleeping abundantly. A few days later she was still asymptomatic.

### Case Report 6

This case report provides an example of needle prick therapy for abdominal pain in an elderly woman. The woman was experiencing persistent abdominal pain accompanied with a normal hematological picture. Relief was achieved after the completion of bilateral infiltration at both T6-7 and L3-4. Her pain continued to recur at approximately weekly intervals. After a negative colon evaluation and multiple consultations with a surgeon and gastroenterologist, her third hospitalization included an endoscopic evaluation that revealed both duodenal and gastric ulcers.

### Case Report 7

This case report provides an example of needle prick therapy for discomfort of a lower extremity. Specifically, an elderly retiree suffered an acute onset of severe discomfort and cramps in the right lower extremity. This episode was resolved by two weeks of bed rest. Months later a more severe attack occurred which was not relieved by bed rest and required use of crutches to ambulate a few steps. Infiltration at unilateral L3-4 allowed him to walk without crutches and attend an 8-hour outing. While on his feet the rest of the day, his only complaint was that of the sensation of numbness streaking down his affected extremity. These streaking numb sensations were gone by the next day and did not accompany the second treatment. A subsequent MRI revealed a 60% stenosis of the spinal cord by a protrusion at L3-4. One month later, a second session of infiltration converted an acute episode into a bearable chronic state. Later, without a previous history, there was the development of digestive symptoms and hypertensive readings in the neighborhood of 220/110. The hypertension was controlled by medications and after two years the digestive symptoms disappeared.

While the invention has been described with respect to a limited number of embodiments, those skilled in the art, having benefit of this disclosure, will appreciate that other embodiments can be devised which do not depart from the scope of the invention as disclosed herein.

1. A needle device, comprising a rotator portion comprising a plurality of needles, and a handle portion, wherein the device does not comprise a means for delivering a substance.

2. The needle device of claim 1, wherein the needles are spaced about 6 mm apart from each other.

3. The needle device of claim 1, wherein the needles are spaced less than about 6 mm apart from each other.

4. The needle device of claim 1, wherein the needles are spaced more than about 6 mm apart from each other.

5. The needle device of claim 1, wherein the plurality of needles are present in one row.

6. The needle device of claim 1, wherein the plurality of needles are present in more than one row.

7. The needle device of claim 1, wherein the plurality of needles are present on a disposable strip.

8. A method for treating an individual having an anatomical insult, comprising delivering multiple cutaneous needles pricks to skin overlying an area innervated by sensory fibers of one or more nerve roots, using the device of claim 1.

9. The method of claim 8, wherein the individual is treated for pain relief.

10. The method of claim 8, wherein an individual experiencing pain is diagnosed.

11. A needle device, comprising a plurality of needles present on a planar support, wherein the needles are arranged into one or more rows, wherein the device does not comprise a means for delivering a substance.

12. The needle device of claim 11, wherein the needles are spaced about 6 mm apart from each other.

13. The needle device of claim 11, wherein the needles are spaced less than about 6 mm apart from each other.

14. The needle device of claim 11, wherein the needles are spaced more than about 6 mm apart from each other.

15. The needle device of claim 11, wherein the plurality of needles are present in two or more rows.

16. The needle device of claim 11, wherein the plurality of needles are present in more than two rows.

17. The needle device of claim 11, wherein the plurality of needles are present on a disposable strip.

18. A method for treating an individual having an anatomical insult, comprising delivering multiple cutaneous needles pricks to skin overlying an area innervated by sensory fibers of one or more nerve roots, using the device of claim 11.

19. The method of claim 18, wherein the individual is treated for pain relief.

20. The method of claim 18, wherein an individual experiencing pain is diagnosed.
